SEQUENCE LISTING (25 sequences)

SEQ ID NO: 1 - DNA, 849 bp, Homo sapiens
  human B7 superfamily member 1 (B7S1) open reading frame
atggcttccctggggcagatcctcttctggagcataattagcatcatcattattctggctggagcaattgcactcatcattggctttggtatttcagggagacactccatcacagtcactactgtcgcctcagctgggaacattggggaggatggaatccagagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataa

SEQ ID NO: 2 - protein, 282 aa, Homo sapiens
  human B7 superfamily member 1 (B7S1)
MASLGQILFWSIISIIIILAGAIALIIGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 3 - DNA, 852 bp, Mus sp.
  mouse B7 superfamily member 1 (B7S1) open reading frame
atggcttccttggggcagatcatcttttggagtattattaacatcatcatcatcctggctggggccatcgcactcatcattggctttggcatttcaggcaagcacttcatcacggtcacgaccttcacctcagctggaaacattggagaggacgggaccctgagctgcacttttgaacctgacatcaaactcaacggcatcgtcatccagtggctgaaagaaggcatcaaaggtttggtccacgagttcaaagaaggcaaagacgacctctcacagcagcatgagatgttcagaggccgcacagcagtgtttgctgatcaggtggtagttggcaatgcttccctgagactgaaaaacgtgcagctcacggatgctggcacctacacatgttacatccgctcctcaaaaggcaaggggaatgcaaaccttgagtataagaccggagccttcagtatgccagagataaatgtggactataatgccagttcagagagtttacgctgcgaggctcctcggtggttcccccagcccacagtggcctgggcatctcaagttgaccaaggagccaacttctcagaagtctccaacaccagctttgagttgaactctgagaatgtgaccatgaaggtcgtatctgtgctctacaatgtcacaatcaacaacacatactcctgtatgattgaaaacgacattgccaaagccaccggggacatcaaagtgacagattcagaggtcaaaaggcggagtcagctgcagttgctgaactctgggccttccccgtgtgtttcttcttctgcctttgtggctggctgggcactcctatctctctcctgttgcctgatgctaagatga

SEQ ID NO: 4 - protein, 283 aa, Mus sp.
  mouse B7 superfamily member 1 (B7S1)
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVSSSAFVAGWALLSLSCCLMLR

SEQ ID NO: 5 - DNA, 852 bp, Unknown Organism
  Description of Unknown OrganismB7 superfamily member 1 (B7S1) cDNA from unspecified organism
atggcttccttggggcagatcatcttttggagtattattaacatcatcatcatcctggctggggccatcgcactcatcattggctttggcatttcaggcaagcacttcatcacggtcacgaccttcacctcagctggaaacattggagaggacgggaccctgagctgcacttttgaacctgacatcaaactcaacggcatcgtcatccagtggctgaaagaaggcatcaaaggtttggtccacgagttcaaagaaggcaaagacgacctctcacagcagcatgagatgttcagaggccgcacagcagtgtttgctgatcaggtggtagttggcaatgcttccctgagactgaaaaacgtgcagctcacggatgctggcacctacacatgttacatccgcacctcaaaaggcaaagggaatgcaaaccttgagtataagaccggagccttcagtatgccagagataaatgtggactataatgccagttcagagagtttacgctgcgaggctcctcggtggttcccccagcccacagtggcctgggcatctcaagtcgaccaaggagccaatttctcagaagtctccaacaccagctttgagttgaactctgagaatgtgaccatgaaggtcgtatctgtgctctacaatgtcacaatcaacaacacatactcctgtatgattgaaaacgacattgccaaagccaccggggacatcaaagtgacagattcagaggtcaaaaggcggagtcagctgcagctgctcaactccgggccttccccgtgtgttttttcttctgcctttgcggctggctgggcgctcctatctctctcctgttgcctgatgctaagatga

SEQ ID NO: 6 - protein, 283 aa, Mus sp.
  deduced amin acid sequence of mouse B7 superfamily member 1 (B7S1) cDNA
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFAAGWALLSLSCCLMLR

SEQ ID NO: 7 - protein, 7 aa, Artificial Sequence
  Description of Artificial SequenceB7 superfamily member 1 (B7S1) consensus peptide
MASLGQI

SEQ ID NO: 8 - protein, 5 aa, Artificial Sequence
  Description of Artificial SequenceB7 superfamily member 1 (B7S1) consensus peptide
FWSII

SEQ ID NO: 9 - protein, 19 aa, Artificial Sequence
  Description of Artificial SequenceB7 superfamily member 1 (B7S1) consensus peptide
IIIILAGAIALIIGFGISG

SEQ ID NO: 10 - protein, 5 aa, Artificial Sequence
  Description of Artificial SequenceB7 superfamily member 1 (B7S1) consensus peptide
ITVTT

SEQ ID NO: 11 - protein, 9 aa, Artificial Sequence
  Description of Artificial SequenceB7 superfamily member 1 (B7S1) consensus peptide
SAGNIGEDG

SEQ ID NO: 12 - protein, 10 aa, Artificial Sequence
  Description of Artificial SequenceB7 superfamily member 1 (B7S1) consensus peptide
SCTFEPDIKL

SEQ ID NO: 13 - protein, 9 aa, Artificial Sequence
  Description of Artificial SequenceB7 superfamily member 1 (B7S1) consensus peptide
IVIQWLKEG

SEQ ID NO: 14 - protein, 11 aa, Artificial Sequence
  Description of Artificial SequenceB7 superfamily member 1 (B7S1) consensus peptide
GLVHEFKEGKD

SEQ ID NO: 15 - protein, 14 aa, Artificial Sequence
  Description of Artificial SequenceB7 superfamily member 1 (B7S1) consensus peptide
EMFRGRTAVFADQV

SEQ ID NO: 16 - protein, 19 aa, Artificial Sequence
  Description of Artificial SequenceB7 superfamily member 1 (B7S1) consensus peptide
VGNASLRLKNVQLTDAGTY

SEQ ID NO: 17 - protein, 21 aa, Artificial Sequence
  Description of Artificial SequenceB7 superfamily member 1 (B7S1) consensus peptide
TSKGKGNANLEYKTGAFSMPE

SEQ ID NO: 18 - protein, 9 aa, Artificial Sequence
  Description of Artificial SequenceB7 superfamily member 1 (B7S1) consensus peptide
NVDYNASSE

SEQ ID NO: 19 - protein, 14 aa, Artificial Sequence
  Description of Artificial SequenceB7 superfamily member 1 (B7S1) consensus peptide
LRCEAPRWFPQPTV

SEQ ID NO: 20 - protein, 61 aa, Artificial Sequence
  Description of Artificial SequenceB7 superfamily member 1 (B7S1) consensus peptide
WASQVDQGANFSEVSNTSFELNSENVTMKVVSVLYNVTINNTYSCMIENDIAKATGDIKVT

SEQ ID NO: 21 - protein, 4 aa, Artificial Sequence
  Description of Artificial SequenceB7 superfamily member 1 (B7S1) consensus peptide
KRRS

SEQ ID NO: 22 - protein, 6 aa, Artificial Sequence
  Description of Artificial SequenceB7 superfamily member 1 (B7S1) consensus peptide
LQLLNS

SEQ ID NO: 23 - protein, 4 aa, Artificial Sequence
  Description of Artificial SequenceB7 superfamily member 1 (B7S1) consensus peptide
WALL

SEQ ID NO: 24 - protein, 7 aa, Artificial Sequence
  Description of Artificial Sequenceconserved motif in first Ig-like domain
  MOD_RES (1)..(7): Xaa = any amino acid
DXGXYXC

SEQ ID NO: 25 - protein, 5 aa, Artificial Sequence
  Description of Artificial Sequencepeptide linker
GGGGS

(19) United States
(12) Patent Application Publication
(10) Pub. No.: US 2008/0008706 A1
(43) Pub. Date: Jan. 10, 2008

(54) B7S1: an immune modulator

(75) Inventors: Chen Dong, Houston, TX (US); Durbaka V.R. Pradas, Osaka (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The University of Washington, Seattle, WA (US)

(21) Appl. No.: 11/804,398

(22) Filed: May 17, 2007

Related U.S. Application Data
(63) Continuation of application No. 10/871,696, filed on Jun. 16, 2004, now abandoned.
(60) Provisional application No. 60/479,244, filed on Jun. 16, 2003.

(51) Int. Cl. A61K 39/395; A61K 38/00; A61P 35/00

(52) U.S. Cl. 424/133.1; 514/2

(57) ABSTRACT

The invention provides B7S1 nucleic acid, B7S1 polypeptides, and antibodies that bind B7S1 polypeptides. B7S1 sequences can be used, e.g., to screen for modulators of B7S1 activity. Modulators, e.g., antibodies or small molecules, can be used for the treatment of disease that involve an immune response.

### CROSS-REFERENCES TO RELATED APPLICATIONS

This application is a continuation of patent application Ser. No. 10/871,696 filed Jun. 16, 2004, which claims benefit of U.S. provisional application No. 60/479,244, filed Jun. 16, 2003, both of which are incorporated by reference herein.

### STATEMENT AS TO RIGHTS TO INVENTIONS MADE UNDER FEDERALLY SPONSORED RESEARCH OR DEVELOPMENT

This invention was made with Government support under Grant No. A1 50746, awarded by the National Institutes of Health. The Government has certain rights in this invention.

### BACKGROUND OF THE INVENTION

T lymphocytes are key mediators in immune responses and in various immune diseases. T cell activation requires two signals: one via TcR recognition of antigenic peptides presented by MHC molecules and the other from costimulatory molecules on the antigen-presenting cells (APC). The best-characterized co-stimulatory molecules are CD80 and CD86, also known as B7.1 and B7.2, respectively, which are expressed by professional APC as a result of innate activation. The receptors for CD80 and CD86 are CD28 and CTLA4. CD28 is expressed by naïve and activated T cells, and plays a major role in T cell activation. Mice lacking CD28 or both CD80 and CD86 are impaired in T cell immune responses in vitro and in vivo. CTLA4, on the other hand, is induced after T cell activation and binds to the same ligands with a higher affinity. CTLA4 carries an ITIM motif in its cytoplasmic region and functions as a negative regulator of T cell activation; CTLA4 knockout mice develop profound autoimmune diseases. Therefore, CD28 and CTLA4 engaged by CD80 and CD86 molecules on APC, play essential roles in maintaining the threshold of T cell activation.

In the past several years, the number of identified members in both the B7 ligand and the corresponding CD28 receptor families has increased. Inducible costimulator (ICOS), a third member of the CD28 family, is expressed on activated but not naïve T cells, and recognizes its own ligand B7h (also named as B7RP-1 etc). B7h is constitutively expressed in certain APC such as B cells and macrophages, and can be induced in non-lymphoid tissues and cells by inflammatory stimuli. We recently generated ICOS-deficient mice and identified ICOS as an important regulator of T cell activation, differentiation and function. PD-1, another ITIM-containing receptor expressed on activated T cells, binds to B7-H1/PDL1 and PDL2/B7DC that are broadly expressed in APC and non-lymphoid tissues. PD-1 plays an important role in maintaining immune tolerance as PD-1-deficient mice develop multiple autoimmune diseases on different genetic backgrounds. B7-H3 is the newest addition to the B7 family whose receptor has not been identified. B7-H3 was first reported to be expressed by human dendritic cells and to stimulate human T cell proliferation and IFNγ production. Recently, we identified the mouse B7-H3 homologue that is broadly expressed in lymphoid and non-lymphoid tissues; a soluble mouse B7-H3-Ig fusion protein binds to activated but not naïve T cells.

Although these costimulators have been shown to play important immune regulatory functions, exploration of their modulation for treatment of immune diseases has not been successful. Thus, additional targets and further understanding of function is required in order to be able to adequately exploit B7 molecules and their regulation of the immune system. This invention addresses that need.

### BRIEF SUMMARY OF THE INVENTION

The present invention is based on the discovery of a novel member of the B7 family, named B7 superfamily member 1 (B7S1). B7S1 is expressed on professional APC and is broadly distributed in non-lymphoid tissues. It functions as a negative costimulator and regulates the threshold of T cell activation. The invention thus provides B7S1 polypeptides and methods of using such polypeptides and cells expressing them to identifying antagonists and agonists of B7S1 activity. The invention further provides methods of modulating T-cell activation by administering antagonists and agonist of B7S1 activity.

In on aspect, the invention provides a method of identifying a modulator of B7S1 activity, the method comprising: contacting a B7S1 polypeptide: (a) comprising an amino acid sequence having at least 80%, typically 85%, or 90% identity to amino acids 43-254 of SEQ ID NO:2; or (b) comprising at least 50, typically 100 or 200 contiguous amino acid residues of SEQ ID NO:2 or 4; with a candidate compound; and selecting a compound that binds to the polypeptide. Typically, the method further comprising assessing T-cell activation in the presence of the compound; and selecting a compound that alters the level of T-cell activation. In some embodiments, the compound is, an antibody or small molecule. In other embodiments, the polypeptide comprises amino acid residues 43-254 of SEQ ID NO:2 or SEQ ID NO:4, or comprises the amino acid sequence set forth in SEQ ID NO:2 or SEQ ID NO:4. The B7S1 polypeptide can be recombinant. Further, it the B7S1 polypeptide can be expressed on a cell. In some embodiments, the cell comprises an expression vector that expresses the B7S1 polypeptide.

In another aspect, the invention provides a method of identifying a modulator of B7S1 activity, the method comprising: contacting a T-cell with a candidate compound, e.g. an antibody or small molecule, and an isolated polypeptide: (a) comprising an amino acid sequence having at least 80%, typically 85%, or 90% identity to amino acids 43-254 SEQ ID NO:2; or (B) comprising at least 50, preferably at least 100 or 200 contiguous residues of SEQ ID NO: 2 or 4; determining the level of T-cell activation in comparison to the level of T-cell activation in the absence of the compound; and selecting a compound that alters the level of T-cell activation. Alternatively, the invention provides a method of identifying a modulator of B7S1 activity, the method comprising: contacting a T-cell with a candidate compound, e.g., an antibody or small molecule, that binds a B7S1 polypeptide as set forth above; determining the level of T-cell activation in comparison to the level of T-cell activation in the absence of the compound; and selecting a compound that alters the level of T-cell activation. In some embodiments of the methods, the polypeptide comprises amino acids 43-254 of SEQ ID NO:2 or SEQ ID NO:4. In further embodiments, the polypeptide comprises SEQ ID NO:2 or SEQ ID NO:4.

In another aspect, the invention provides a method of enhancing T-cell activation, the method comprising contacting a T-cell with an agent, e.g., an antibody or small molecule, that inhibits binding of B7S1to the T-cell. In some embodiments, the antibody or small molelcule specifically binds B7S1. The antibody may be a monoclonal antibody. In some embodiments, the monoclonal antibody is a chimeric antibody or humanized antibody. The antibody may also be a human antibody. In some embodiments, the antibody is a single chain Fv fragment (scFv). The agent may be administered to a patient having an infectious disease or cancer.

In further embodiments, the agent is an siRNA, anti-sense RNA, or ribozyme that binds to a nucleic acid sequence encoding B7S1.

The invention also provides a method of inhibiting T-cell activation, the method comprising administering a polypeptide: (a) comprising an amino acid sequence having at least 80%, typically 85%, or 90% identity to amino acid residues 43-254 SEQ ID NO:2; or (b) comprising at least 50, typically at least 100 or 200 contiguous amino acids of amino acids 43-254 of SEQ ID NO:2 or 4. In some embodiments, the polypeptide comprises amino acid residues 43-254 SEQ ID NO:2 or SEQ ID NO:4. The polypeptide may, e.g. be B7S1-Ig. In other embodiments, the method comprises administering an expression vector comprising a nucleic acid sequence encoding the polypeptide. In some embodiments, the polypeptide is administered to a patient having an autoimmune disease.

In another aspect, the invention provides a method of inhibiting T-cell activation. The method comprises administering an inhibitor of T-cell activation identified in accordance with the methods described above. In some embodiments, the method of identifying the inhibitor comprising assessing binding of a candidate inhibitor to a B7S1 polypeptide and assessing the effects of the compound on T-cell activation. Such an inhibitor can be a small molecule, a peptide, or an antibody that mimics B7S1 activity. In some embodiments, the inhibitor is administered to a patient having an autoimmune disease.

The invention also provides a B7S1 polypeptide and expression vector comprising a nucleic acid sequence encoding the polypeptide, where the polypeptide: (a) has at least 80%, typically 85%, or 90% identity to amino acids 43-254 of SEQ ID NO:2 or SEQ ID NO:4; or (b) comprises at least 50, typically at least 100, or 200 contiguous residues of amino acids 43-254 of SEQ ID NO:2 or SEQ ID NO:4. In some embodiments, polypeptide comprises amino acid residues 43-254 of SEQ ID NO:2 or SEQ ID NO:4. Such a polypeptide, may, e.g., comprises the amino acid sequence set forth in SEQ ID NO:2 or SEQ ID NO:4.

In another aspect, the invention provides a cell comprising an expression vector as set forth above.

The invention also provides antibodies that bind to B7S1 polypeptides, e.g., SEQ ID NO:2 and/or SEQ ID NO:4, or a domain or fragment thereof. The antibody can be, e.g., a monoclonal antibody, a chimeric antibody, a humanized antibody, a human antibody, or an scFV. In some embodiments, the antibody is an antibody that blocks B7S1 -mediated inhibition of T-cell activation, e.g., clone 54. In other embodiments, the antibody competes with clone 54 for binding to B7S1 or binds to the same epitope as B7S1.

### BRIEF DESCRIPTION OF THE DRAWINGS

FIG. 1. Identification of B7S1as a novel member of the B7 superfamily. (A). Nucleotide sequence of B7S1 cDNA (SEQ ID NO:5) encompassing the open reading frame. (B). Alignment of deduced amino acid sequence of mouse B7S1 cDNA (SEQ ID NO:6) with its human homologue (SEQ ID NO:2). The N-terminal leader peptide and C-terminal hydrophobic regions are indicated by straight lines; Ig-like domains, by dotted lines. The residues conserved (consensus peptides=SEQ ID NOS:7-23) in all members of the B7 family are indicated by underlines. (C). Phylogenic analysis of the B7 family members. The scores at each branch represent the degree of sequence variability between proteins (0 for identical sequences). Percentage of amino acid identity between B7S1 protein and the other members of the B7 family is indicated.

FIG. 2. (A). Generation of monoclonal antibodies to mouse B7S1. Supernatants from anti-B7S1 hybridoma clones 9 and 54 were used to stain B7S1-, mock- or B7-H3-transfected 293 cells, and the staining was revealed by an anti-rat IgG-FITC. (B) B7S1 expression is sensitive to PI-PLC treatment. 293 cells transfected with a B7S1 expression vector (for B7S1) or EL-4 cells (for Thy1 and ICOS) were treated with PI-PLC at 37 C for 30 minutes. Cells with (PI-PLC) or without the treatment (NT) were stained with antibodies to these antigens. Mean fluorescence (MF) of each staining is indicated.

FIG. 3. Expression of B7S1in tissues and by immune cells. (A). A PCR fragment consisting of two Ig-like domains of the mouse B7S1 gene was used to hybridize mouse tissue Northern blot (Seegene, Inc., Korea). (B). Expression of B7S1 by thymocytes, spleen cells and peritoneal macrophages. Cells, were stained with a biotinylated anti-B7S I antibody in conjunction with other indicated markers.

FIG. 4. B7S1-Ig binds to activated T cells. Lymph node cells from a C57BL/6 mouse were activated with ConA for 48 hours, and cells before and after activation were analyzed for B7S1-Ig binding together with antibodies for CD4 and CD8.

FIG. 5. B7S1-Ig inhibits T cell proliferation and IL-2 production. (A-C). CD4 T cells isolated from C57BL/6 (A) or OT-II (B-C) mice were treated with indicated doses of various stimuli, and T cell proliferation measured by ³H-thymidine incorporation. (D). CD4 T cells from C57BL/6 mice were stimulated with indicated doses of anti-CD3 with or without anti-CD28 (2 μg/ml) in the presence or absence of B7S1-Ig and their proliferation measured. (E). OT-II T cells were treated by indicated means for 24 hours and IL-2 expression measured by ELISA. (F). Exogenous IL-2 restored proliferation by B7S1 -treated OT-IL cells. OT-II cells were treated as in B at the presence of exogenous IL-2 (30 units/ml) and cell proliferation assayed.

FIG. 6. Anti-B7S1 blocking antibody enhanced T cell proliferation and IL-2 production in vitro. (A). Biotinylated B7S1-Ig was incubated with a rat control Ig (no blocking) or clone 54 anti-B7S1 (blocking) before staining with ConA-activated mouse lymph nodes cells. (B-C). Spleen cells from C57BL/6 mice were incubated with indicated doses of anti-CD3 at the presence of 5 μg/ml control rat IgG or purified clone 54 antibody. Cell proliferation (B) was measured by ³H-thymidine uptake after 72 hours and IL-2 (C) assayed by ELISA 24 hours after the treatment.

FIG. 7. Anti-B7S1 blocking antibody enhanced T-dependent immune responses and EAE disease in vivo. (A-C). C57BL/6 mice (3 in each group) immunized with KLH in CFA were treated with a rat control Ig or anti-B7S1 blocking antibody. Eight days after the immunization, experimental mice were sacrificed and anti-KLH serum IgM was measured by ELISA (A). Spleen cells from immunized mice were restimulated in vitro with or without KLH and T cell proliferation (B) and IL-2 production (C) was measured. (D-E). C57BL/6 mice (5 mice in each group) immunized with MOG peptide to induced EAE were treated with a rat control Ig or anti-B7S1 blocking antibody. (D). EAE disease in these mice was scored. The result shown is a representative of two independent experiments with similar results. (E). Mononuclear cells in CNS from mice with EAE were typed by staining with anti-CD4, CD8 or CD11b.

### DETAILED DESCRIPTION OF THE INVENTION

The present invention is based on the discovery of a novel member of the B7 family, named B7 superfamily member 1 (B7S1). B7S1 is expressed on professional APC and is broadly distributed in non-lymphoid tissues. Its expression on B cells is downregulated following their activation. B7S1 is a novel negative costimulator and regulates the threshold of T cell activation.

### Definitions

The terms “B7S1” therefore refers to nucleic acid and polypeptide polymorphic variants, alleles, mutants, and interspecies homologs and domains thereof that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a full length protein, or a window of at least about 25, 50, 100, or 200 or more amino acids, to a sequence of SEQ ID NO:2 or SEQ ID NO:4; (2) bind to antibodies raised against an immunogen comprising an amino acid sequence of SEQ ID NO:2 or SEQ ID NO:4, and conservatively modified variants thereof, (3) have at least 15 contiguous amino acids, more often, at least 20, 25, 30, 35, 40, 50, 100, or 200 contiguous amino acids, of SEQ ID NO:2 or SEQ ID NO:4; (4) specifically hybridize (with a size of at least about 100, preferably at least about 200, or 500 nucleotides) under stringent hybridization conditions to a sequence of SEQ ID NO: 1 or SEQ ID NO:3, and conservatively modified variants thereof; (5) have a nucleic acid sequence that has greater than about 95%, preferably greater than about 96%, 97%, 98%, 99%, or higher nucleotide sequence identity, preferably over a region of at least about 50, 100, 200, 500, 800, or more nucleotides, to SEQ ID NO:1 or SEQ ID NO:3; or (6) are amplified by primers that specifically hybridize under stringent conditions to SEQ ID NO: 1 or SEQ ID NO:3. This term also refers to a domain of a B7S1, as described above, or a fusion protein comprising a domain of a B7S1 linked to a heterologous protein. A B7S1 polynucleotide or polypeptide sequence of the invention is typically from a mammal including, but not limited to, human, mouse, rat, hamster, cow, pig, horse, sheep, or any mammal. A “B7S1 polynucleotide” and a “B7S1 polypeptide,” are both either naturally occurring or recombinant.

“Extracellular domain” refers to the domain of a B7S1 that protrudes from the cellular membrane and often binds to an extracellular ligand. This domain is often useful for in vitro ligand binding assays, both soluble and solid phase. The domain may be joined to another compound, e.g., another polypeptide, such as an Ig molecule. The extracellular domain can be identified based on known parameters, e.g., structural analyses, or by sequence similarity to known B7S1 polypeptide sequences, e.g., SEQ ID NO:2 or 4. The extracellular domain is from about amino acid residue 43 to about amino acid residue 254 of SEQ ID NOs. 2 and 4. As appreciated by one of skill in the art, the extracellular domain may be somewhat shorter in length, e.g., comprises at least 175, 180, 185, 190, 195, or 200 contiguous amino acids of the region encompassed by amino acid residues 43-254 of SEQ ID NOs 2 and 4. An extracellular domain typically has at least 70%, often 75%, 80%, 85%, 90%, 95%, 96%, 97%, 98%, or 99% identity with amino acids residues 43-254 of SEQ ID NO:2 or 4.

The “activity” of a B7S1 polypeptide can be determined using a variety of assays typically assays that reflect T-cell activation. Such assays include, but are not limited to, binding to activated T-cells, proliferation of T-cells, production of IL-2, and assessment of JunB activity or expression. Exemplary assays are provided in the “Examples” section.

“T-cell activation” refers to the ability of a T-cell to respond to an antigenic epitope or a non-specific T-cell mitogen that is presented to the T-cell. Activation of a T-cell is characterized by proliferation, production of IL-2, and differentiation into effector cells.

“Inhibitors,” “mimics,” and “modulators” of B7S1refer to inhibitory, activating, or modulating molecules that influence, either positively or negatively, B7S1 activity, e.g., B7SI1-mediated inhibition of T-cell activation. Such modulators can be identified using in vitro and in vivo assays. Modulating molecules, also referred to herein as compounds, include polypeptides, antibodies, amino acids, nucleotides, lipids, carbohydrates, or any organic or inorganic molecule. B7S1 inhibitors are compounds that partially or totally block, decrease, prevent, delay, or inhibit B7S1-induced inhibition of T-cell activation. Such inhibitors typically bind to a B7S1 polypeptide or polynucleotide sequence. A B7S1 “mimic” has the activity of a B7S1 polypeptide or is able to enhance the activity of a B7S1 polypeptide, i.e., inhibit T-cell activation. Such compounds include analogs of B7S1 and molecules that activate T-cells and compete with B7S1 in a T-cell activation assay, e.g., T-cell proliferation, IL-2 induction, JunB induction, and the like.

As used herein, “agonist” refers to a compound that mimics the activity of B7S1, i.e., it inhibits T-cell activation. An “antagonist” refers to a compound that inhibits the activity of B7S1. For example, an antibody that blocks B7S1-mediated inhibition of T-cell activation, is considered an antagonist in the context of this invention.

Samples or assays comprising B7S1 polypeptides that are treated with a potential modulator are compared to control samples without the modulator to examine the extent of activity relative to the B7S1 polypeptide. Control samples (untreated with modulators) are assigned a relative activity value of 100%. Inhibition of B7S1 activity is achieved when the activity value relative to the control is about 80%, preferably 50%, more preferably 25-0%. As appreciated by one of skill in the art, B7S1 polypeptides such as those set forth in SEQ ID NOs: 2 and 4 inhibit T-cell activation. Accordingly, inhibition of B7S1 activity results in enhancement of T-cell activation, i.e., T-cell activation is increased relative to a control that does not contain an inhibitor of B7Sa activity. Thus, although B7S1 activity is inhibited, i.e., decreases, assay readout may show an absolute increase in activity of the assay parameter, e.g., T-cell proliferation or IL-2 production, which reflects inhibited B7S1 activity. A “mimic” or “agonist” of a B7S1 protein, e.g., a variant of a B7S1 as described herein, a peptide, or small molecule, shows an activity that is essentially equal to that of B7S1, although in some instances may be greater, e.g., 110%, 150%, or higher relative to the activity of B7S1.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol :5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The positions of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M. -P. IMGT, the international ImMunoGeneTics database. Nucleic Acids Res. Jan 1 ;29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

References to “V_{H}” or a “VH” refer to the variable region of an immunoglobulin heavy chain of an antibody, including the heavy chain of an Fv, scFv , or Fab. References to “V_{L}” or a “VL” refer to the variable region of an immunoglobulin light chain, including the light chain of an Fv, scFv , dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The term “fully human antibody” refers to an immunoglobulin comprising human variable regions in addition to human framework and constant regions. Such antibodies can be produced using various techniques known in the art. For example in vitro methods involve use of recombinant libraries of human antibody fragments displayed on bacteriophage (e.g., McCafferty et al., 1990, Nature 348:552-554; Hoogenboom & Winter, J. Mol. Biol. 227:381 (1991); and Marks et al., J. Mol. Biol. 222:581 (1991)), yeast cells (Boder and Wittrup, 1997, Nat Biotechnol 15:553-557), or ribosomes (Hanes and Pluckthun, 1997, Proc Natl Acad Sci USA 94:4937-4942). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 6,150,584, 5,545,807; 5,545,806; 5,569,825; 5,625,126; 5,633,425; 5,661,016, and in the following scientific publications: (e.g., Jakobavits, Adv Drug Deliv Rev. 31:33-42(1998), Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term ” binding specificity,” “specifically binds to an antibody” or “specifically immunoreactive with,” refers to a binding reaction which is determinative of the presence of a B7S1 polypeptide in the presence of a heterogeneous population of proteins and other biologics. Thus, under designated immunoassay conditions, the specified antibodies bind to a B7S1 at least two times the background and more typically more than 10 to 100 times background.

Specific binding of an antibody to a protein under such conditions requires an antibody that is selected for its specificity for a particular protein. For example, antibodies raised to a particular protein, polymorphic variants, alleles, orthologs, and conservatively modified variants, or splice variants, or portions thereof, can be selected to obtain only those antibodies that are specifically immunoreactive with B7S1 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the TUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e:g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

An “expression vector” contains an expression cassette that includes all the elements required for the expression of the B7S1-encoding nucleic acid in host cells. A typical expression cassette thus contains a promoter operably linked to the nucleic acid sequence encoding a B7S1 polypeptide or fragment thereof, and signals required for efficient polyadenylation of the transcript, ribosome binding sites, and translation termination. Additional elements of the cassette may include enhancers and, if genomic DNA is used as the structural gene, introns with functional splice donor and acceptor sites.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

Introduction

The current invention is based on the discovery of a new polypeptide that inhibits T-cell activion. Accordingly, B7S1 conservative modifications, or variants thereof, may be used to modulate T-cell activation activity and for the treatment of diseases or conditions for which it is desirable to suppress T-cell activity, e.g., autoimmune disorders. Further, B7S1 sequences may be used to identify compounds that modulate B7S1 activity, e.g., to identify inhibitors of B7S1 activity such as antibodies or small molecules. Such modulators may be administered in diseases or states for which it is desirable to enhance an immune response, e.g., for example, cancer or infectious disease.

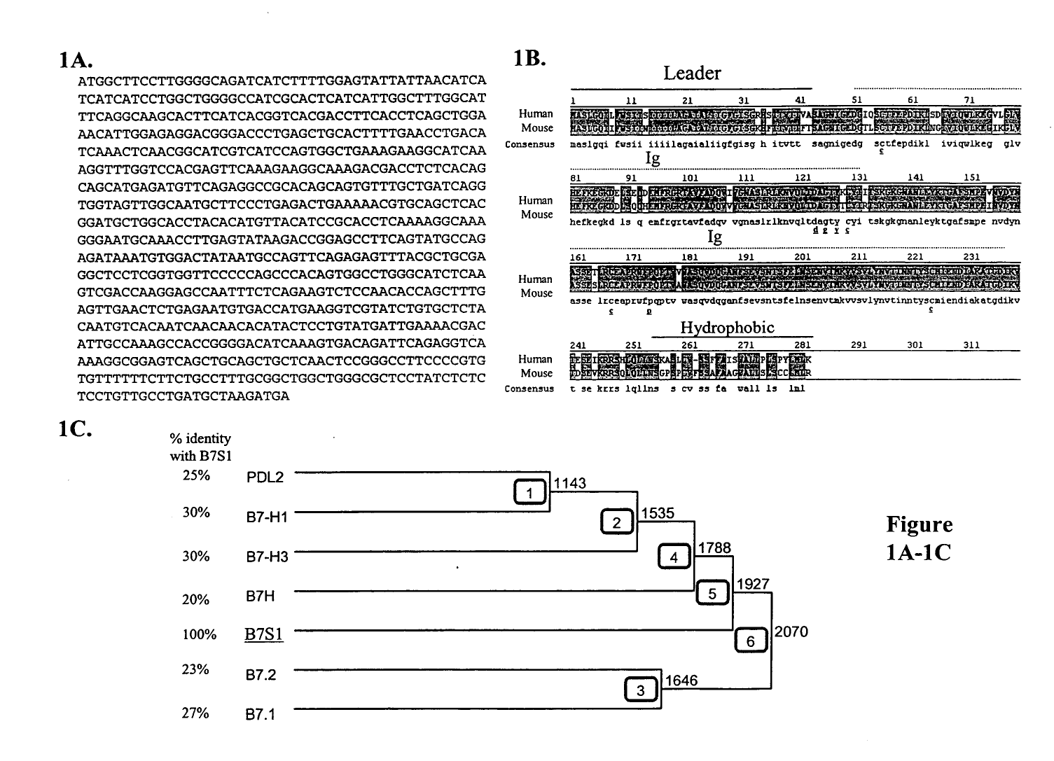

The present invention thus provides B7S1 polypeptide and nucleic acid sequences. Exemplary B7S1 polypeptides sequences are set forth in SEQ ID NOS:2 and 4. Human and mouse B7S1 polypeptides are about 87% identical over their length. The sequences are over 90% identical over the extracellular domain, about amino acid 43 to about amino acid 254 of SEQ ID NOS:2 and 4. Full-length B7S1 sequences contain an N-terminal hydrophobic regions that can serve as a leader peptide, two immunoglobulin (Ig)-like domains, and a hydrophobic C-terminus. This protein is similar to existing B7 family members, with 20%-30% identity. Important cysteine residues as well as the DxGxYxC motif (SEQ ID NO:24) in the first Ig-like domain are conserved in these proteins (see, e.g., FIG. 1B).

Related B7S1 genes, e.g., homologs from other species or variants, should share at least about 70%, 80%, 90%, or greater, amino acid identity over a amino acid region at least about 25 amino acids in length, optionally 50 to 100 or 200 amino acids in length. Antibodies that bind specifically to a B7S1 or a conserved region thereof can also be used to identify alleles, interspecies homologs, and variants.

The B7S1 polypeptides of the invention include domains of a full-length B7S1, e.g., the extracellular domain. Such a domain can be used either alone or joined to a heterologous protein, in screening assays to identify modulators of B7S1 or may be administered therapeutically for the treatment of immune disorders or to enhance the immune response.

B7S1 is expressed in most professional antigen-presenting cells, including bone-marrow-derived dendritic cells, peritoneal macrophages and B cells. Its expression is downregulated by multiple stimuli. Evaluation of expression in tissues thus shows expression in lymphoid tissues, e.g., thymus and spleen, as well as in nonlymphoid tissues.

The invention therefore provides B7S1 nucleic acid and polypeptide sequences, antibodies that bind B7S1 and methods of screening for modulators of B7S1 activity. Isolation and expression of nucleic acids encoding B7S1.

This invention relies on routine techniques in the field of recombinant genetics, e.g., expression techniques. Basic texts disclosing the general methods of use in this invention include Sambrook & Russell, Molecular Cloning, A Laboratory Manual (3rd Ed, 2001); Kriegler, Gene Transfer and Expression: A Laboratory Manual (1990); and Current Protocols in Molecular Biology (Ausubel et al., eds., 1994-2004 update). Methods that are used to produce B7S1 polypeptides for use in the invention may also be employed to produce modulators, e.g, B7S1 inhibitors that are polypeptides.

In general, the nucleic acid sequences encoding B7S1 polypeptides and related nucleic acid sequence homologs are cloned from cDNA and genomic DNA libraries by hybridization with a probe, or isolated using amplification techniques with oligonucleotide primers, and verified by sequencing. For example, B7S1 sequences are typically isolated from mammalian nucleic acid (genomic or cDNA) libraries by hybridizing with a nucleic acid probe, the sequence of which can be derived from SEQ ID NO:1 or SEQ ID NO:3, or by using an antibody to screen an expression library. Suitable tissues from which B7S1 RNA and cDNA can be isolated include, e.g., lymphoid tissues or cells and antigen-producing cells.

Amplification techniques using primers can also be used to amplify and isolate B7S1 nucleic acids from DNA or RNA. Suitable primers can be designed using criteria well known in the art (see, e.g., Dieffenfach & Dveksler, PCR Primer: A Laboratory Manual (1995)). These primers can be used, e.g., to amplify either a full length sequence or a fragment thereof.

Synthetic oligonucleotides can also be used to construct recombinant B7S1 genes for use as probes or for expression of protein, for example, by using a series of overlapping oligonucleotides. Alternatively, amplification techniques can be used with precise primers to amplify a specific subsequence of the B7S1 nucleic acid. The specific subsequence is then ligated into an expression vector.

The nucleic acid encoding a B7S1 is typically cloned into intermediate vectors before transformation into prokaryotic or eukaryotic cells for replication and/or expression. These intermediate vectors are typically prokaryote vectors, e.g., plasmids, or shuttle vectors.

Optionally, nucleic acids encoding chimeric proteins comprising B7S1 or domains thereof can be made according to standard techniques. For example, a domain such as an extracellular domain or a glycosyl phosphatidylinositol linkage, which anchors B7S1 to the cell membrane, can be covalently linked to a heterologous protein. For example, an extracellular domain can be linked to an Ig polypeptide, as exemplified in the Examples section.

Expression of B7S1

To obtain expression of a cloned gene or nucleic acid, such as cDNAs encoding a B7S1 polypeptide or fragment thereof, one typically subclones a nucleic acid sequence encoding the protein of interest into an expression vector that contains a promoter to direct transcription, a transcription/translation terminator, and additional components such as a ribosome binding site for translational initiation. Suitable bacterial expression systems are and described, e.g., in Sambrook & Russell and Ausubel et al. Bacterial expression systems for expressing the protein are available in, e.g., E. coli, Bacillus sp., and Salmonella (Palva et al., Gene 22:229-235 (1983); Mosbach et al., Nature 302:543-545 (1983). Such expression systems are commercially available. Eukaryotic expression systems for mammalian cells, yeast, and insect cells are well known in the art and are also commercially available and well known in the art. Viral expression systems, includen adenoviral vectors, adeno-associated vectors, retroviral vectors, as well as many other viral vectors are additionally well known and available commercially.

The particular expression vector used to transport the genetic information into the cell is not particularly critical. Any of the conventional vectors used for expression in eukaryotic or prokaryotic cells may be used. Standard bacterial expression vectors include plasmids such as pBR322 and PUC-based plasmids and fusion expression systems such as GST and LacZ. Epitope tags can also be added to recombinant proteins to provide convenient methods of isolation, e.g., c-myc or his tags.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a B7S1-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of B7S1 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Russell & Sambrook, supra). It is only necessary that the particular genetic engineering procedure used be capable of successfully introducing at least one gene into the host cell capable of expressing a B7S1.

After the expression vector is introduced into the cells, the transfected cells are cultured under conditions favoring expression of a B7S1, which is recovered from the culture using standard techniques identified below.

Transgenic animals, including knockout transgenic animals, that include additional copies of a B7S1 and/or altered or mutated B7S1 transgenes can also be generated. A “transgenic animal” refers to any animal (e.g. mouse, rat, pig, bird, or an amphibian), preferably a non-human mammal, in which one or more cells contain heterologous nucleic acid introduced using transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

In other embodiments, transgenic animals are produced in which expression of B7S1 is silenced. Gene knockout by homologous recombination is a method that is commonly used to generate transgenic animals. Transgenic mice can be derived using methodology known to those of skill in the art, see, e.g., Hogan et al., Manipulating the Mouse Embryo: A Laboratory Manual, (1988); Teratocarcinomas and Embryonic Stem Cells: A Practical Approach, Robertson, ed., (1987); and Capecchi et al., Science 244:1288 (1989).

Either naturally occurring or recombinant B7S1 can be purified for use in functional assays. The protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Russell & Sambrook, supra).

Recombinant proteins are expressed by transformed bacteria or eukaryotic cells such as CHO cells or insect cells in large amounts, typically after promoter induction; but expression can be constitutive. Promoter induction with IPTG is one example of an inducible promoter system. Cells are grown according to standard procedures in the art. Fresh or frozen cells are used for isolation of protein using techniques known in the art (see, e.g., Russell & Sambrook, supra; and Ausubel et al., supra).

A number of procedures can be employed when a recombinant B7S1 is being purified. For example, proteins having established molecular adhesion properties can be reversibly fused to B7S1. With the appropriate ligand, B7S1 can be selectively adsorbed to a purification column and then freed from the column in a relatively pure form. The fused protein is then removed by enzymatic activity. Finally, B7S1 could be purified using immunoaffinity columns.

Production of B7S1 Antibodies

The invention also provides B7S1 antibodies or antibodies that modulate B7S1 activity. A general overview of the applicable technology for generating and identifying antibodies can be found in Harlow & Lane, Antibodies: A Laboratory Manual (1988) and Harlow & Lane, Using Antibodies (1999).

The antibodies of the invention can o be used to detect a B7S1 polypeptide, or cells expressing the polypeptide, e.g., activated T-cells or antigen-presenting cells, using any of a number of well recognized immunological binding assays (see, e.g., U.S. Pat. Nos. 4,366,241; 4,376,110; 4,517,288; and 4,837,168). For a review of the general immunoassays, see also Methods in Cell Biology, Vol. 37, Asai, ed. Academic Press, Inc. New York (1993); Basic and Clinical Immunology 7th Edition, Stites & Terr, eds. (1991).

Methods of producing polyclonal and monoclonal antibodies that react specifically with B7S1 are known to those of skill in the art (see, e.g., Coligan, Current Protocols in Immunology (1991); Harlow & Lane, supra; Goding, Monoclonal Antibodies. Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Such antibodies can be used for therapeutic and diagnostic applications, e.g., in the treatment and/or detection of any of the B7S1-associated diseases or conditions described herein.

A number of B7S1immunogens may be used to produce antibodies specifically reactive with a B7S1. For example, a recombinant B7S1 or an antigenic fragment thereof, e.g., an extracellular domain, can be used. Recombinant protein is the preferred immunogen for the production of monoclonal or polyclonal antibodies. Alternatively, a synthetic peptide derived from the sequences disclosed herein can be used. Typically, such a peptide is conjugated to a carrier protein can be used an immunogen. Naturally occurring protein may also be used either in pure or impure form.

Typically, polyclonal antisera with a titer of 10⁴ or greater are selected and tested for their cross reactivity against non-B7S1 proteins or even other related B7S1 proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Once B7S1 specific antibodies are available, B7S1 polypeptides can be detected by a variety of immunoassay methods, as noted above. For a review of immunological and immunoassay procedures, see e.g., Basic and Clinical Immunology (Stites & Terr eds., supra) and Methods in Cell Biology: Antibodies in Cell Biology, volume 37 (Asai, ed., supra). Such assays include both competitive and noncompetitive assay formats.

An antibody that is specifically reactive with a B7S1 polypeptide, e.g., SEQ ID NO:2, or fragment of B7S1 comprising a subsequence of SEQ ID NO:2, can specifically binds a closely related polypeptide sequence, e.g., SEQ ID NO:4, or a subsequence of SEQ ID NO:2. Such closely related polypeptides, or fragments, typically have at least 85%, often 90%, 95%, or higher sequence identity.

Immunoassays in the competitive binding format can also be used for cross-reactivity determinations. For example, a protein at least partially encoded by SEQ ID NO:2 or SEQ ID NO:4 can be immobilized to a solid support. Proteins (e.g., B7S1 proteins and homologs) are added to the assay that compete for binding of the antisera to the immobilized antigen. The ability of the added proteins to compete for binding of the antisera to the immobilized protein is compared to the ability of B7S1, or a fragment, having the sequence set forth in SEQ ID NO:2 or SEQ ID NO:4 to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, e.g., thought to be perhaps an allele or polymorphic variant of B7S1 to the immunogen protein (i.e., a polypeptide of SEQ ID NO:2 or SEQ ID NO:4). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the immunogen protein that is required to inhibit 50% of binding, then the second protein is said to specifically bind to the polyclonal antibodies generated to a B7S1 immunogen.

B7S1 antibodies may be administered therapeutically. The invention thus also encompasses therapeutic antibodies. Preferably, such antibodies are additionally humanized, using known techniques as described herein. Examples of monoclonal antibodies that may be used therapeutically include the monoclonal antibody clone 54 described in the examples provided herein.

The invention also includes antibodies that compete for binding and/or bind to the same epitope as clone 54. Techniques for identifying such antibodies are known and described, for example, in Harlow & Lane, Using Antibodies, A Laboratory Manual (Cold Spring Harbor Press, 1999). For example, the ability of a particular antibody to recognize the same epitope as another antibody is typically determined by the ability of one antibody to competitively inhibit binding of the second antibody to the antigen. Any of a number of competitive binding assays can be used to measure competition between two antibodies to the same antigen. For example, a sandwich ELISA assay can be used for this purpose. This is carried out by using a capture antibody to coat the surface of a well. A subsaturating concentration of tagged-antigen is then added to the capture surface. This protein will be bound to the antibody through a specific antibody: epitope interaction. After washing a second antibody, which has been covalently linked to a detectable moeity (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

Preferably, antibodies that bind to the same epitope as clone 54 have similar binding affinities. Binding affinity for a target antigen is typically measured or determined by standard antibody-antigen assays, such as Biacore competitive assays, saturation assays, or immunoassays such as ELISA or RIA.

Humanized Antibodies

In some embodiments B7S1 antibodies or B7S1 modulators that are antibodies are or humanized antibodies. As noted above, humanized forms of antibodies are chimeric immunoglobulins in which residues from a complementary determining region (CDR) of human antibody are replaced by residues from a CDR of a non-human species such as mouse, rat or rabbit having the desired specificity, affinity and capacity.

Human antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boemer et al. are also available for the preparation of human monoclonal antibodies (Cole et al., Monoclonal Antibodies and Cancer Therapy, p. 77 (1985) and Boemer et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser (SEQ ID NO:25), preferably 2, 3, 4, 5, or 6 such sequences. However, it is to be appreciated that some amino acid substitutions within the linker can be made. For example, a valine can be substituted for a glycine. Methods of making scFv antibodies have been described. See, Huse et al., supra; Ward et al. supra; and Vaughan et al., supra.

In some embodiments, the antibodies may be bispecific antibodies. Bispecific antibodies are monoclonal, preferably human or humanized, antibodies that have binding specificities for at least two different antigens or that have binding specificities for two epitopes on the same antigen.

Antibody Conjugates

Antibodies of the invention can also comprise other molecules, e.g., an antibody can be conjugated to an effector component. An effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a cytotoxic moiety; activatable moieties, a chemotherapeutic agent; a lipase; an antibiotic; or a radioisotope emitting “hard” e.g., beta radiation.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. In some cases, radioisotopes are used as toxic moieties, as described below. The labels may be incorporated into the nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Assays for Modulators of B7S1

Assays for B 7Activity

The activity of B7S1 polypeptides can be assessed using a variety of in vitro and in vivo assays to determine functional, chemical, and physical effects, e.g., measuring ligand binding, (e.g., radioactive ligand binding), IL-2 production, effects on components of signal transduction, e.g., JunB activity, transcription levels, and the like. Such assays can be used to test for inhibitors and activators that mimic B7S1. In particular, the assays can be used to test for compounds that modulate B7S1-induced T-cell activation, for example, by modulating the binding of B7S1 to a T-cell or by modulating the ability of B7S1 to activate the receptor. Typically in such assays, the test compound is contacted with a T-cell in the presence of B7S1. The B7S1 may be added to the assay before, after, or concurrently with the test compound. The results of the assay, for example, the level of binding, T-cell proliferation, or IL-2 production, is then compared to the level in a control assay that comprises T-cells and B7S1 in the absence of the test compound.

Screening assays of the invention are used to identify modulators that can be used as therapeutic agents, e.g., antibodies to B7S1 that block or enhance its activities, or nucleic acid or small molecules antagonists or mimics of B7S1 activity.

The effects of test compounds upon the function of the B7S1 polypeptide can be measured by examining any of the parameters described above. Any suitable physiological change that affects B7S1 activity can be used to assess the influence of a test compound on B7S1 activity. When the functional consequences are determined using intact cells or animals, one can also measure a variety of effects such as transcriptional changes of components of signal-transductions pathways changed during T-cell activation and changes in cell growth

A B7S1 for use in the assay will be selected from a polypeptide, or domain or fragment, having a sequence of SEQ ID NO:2 or SEQ ID NO:4, or conservatively modified variants thereof. Generally, the polypeptides will be at least 85% identical over a domain or the length of the protein. Thus, the polypeptide will typically be at least 85%, often 90%, or 95% identical over a window of 25, 50, or 100 amino acids. Either a full length B7S1, or a domain thereof, can be covalently linked to a heterologous protein to create a chimeric protein used in the assays described herein. In some embodiments, a B7S1 polypeptide or domain comprises at least 50, often at least 100 or 200 contiguous amino acids of SEQ ID NO:2 or SEQ ID NO:4, or at least 50, often at least 100 or 200 contiguous amino acids of a domain of SEQ ID NO:2 or SEQ ID NO:4, e.g., an extracellular domain, or the membrane anchor domain (see, e.g., FIG. 1).

Modulators of B7S1 activity are tested using B7S1 polypeptides as described above, either recombinant or naturally occurring. The protein can be isolated, expressed in a cell, expressed in a membrane derived from a cell, expressed in tissue or in an animal, either recombinant or naturally occurring. For example, cells of the immune system, transformed cells, or membranes can be used. Modulation is tested using known in vitro or in vivo assays that measure B7S1 binding or T-cell activation. These include the exemplary assay described herein. Activity, e.g., binding, can also be examined in vitro with soluble or solid state reactions, e.g., using an extracellular domain of B7S1.

Binding of a compound to B7S1, or domain can be tested in a number of formats. Binding can be performed in solution, in a bilayer membrane, attached to a solid phase, in a lipid monolayer, or in vesicles. Typically, in an assay of the invention, the binding of a compound to B7S1 is tested directly, using a B7S1 polypeptide. Alternatively, the ability of a compound to affect B7S1 binding to T-cells is measured in the presence of a candidate modulator. Often, competitive assays that measure the ability of a compound to compete with binding of B7S1 to T-cells, or the ability of a known binder to B7S1, e.g., an antibody, to bind B7S1 polypeptides. Binding can be tested by measuring, e.g., changes in spectroscopic characteristics (e.g., fluorescence, absorbance, refractive index), hydrodynamic (e.g., shape) changes, or changes in chromatographic or solubility properties.

In an exemplary assay, T-cell proliferation provides a convenient measure to assess B7S1 activity and the effects of modulators on B7S1 activity. T-cell proliferation can be assessed using assay well known in the art, e.g., measuring tritiated thymidine incorporation, which reflects DNA replication; by measuring cell number, or by measuring other parameters that reflect DNA replication and growth. In such assays, proliferation is measured in response to a mitogen. A T-cell mitogen can be specific, e.g., an antigenic epitope, or a general mitogen, such as an anti-T-cell receptor antibody. T-cell proliferation in response to the mitogen is determined in the presence of B7S1, and/or a B7S1 agonist or antagonist.

In another embodiment, gene expression levels can be measured to assess the effects of a test compound on B7S1 activity. A host cell containing the protein of interest is contacted with a test compound in the presence of B7S1 for a sufficient time to effect any interactions, and then the level of gene expression is measured. The amount of time to effect such interactions may be empirically determined, such as by running a time course and measuring the level of expression as a function of time. The amount of expression may be measured by using any method known to those of skill in the art to be suitable. For example, mRNA expression of the protein of interest, e.g., IL-2, may be detected, or their polypeptide products may be identified using immunoassays. Alternatively, transcription based assays using reporter genes may be used as described in U.S. Pat. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of expression is then compared to the amount of expression in either the same cell in the absence of the test compound, or it may be compared with the amount of expression in a substantially identical cell that lacks the protein of interest. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

Samples that are treated with a candidate modulator are compared to control samples comprising B7S1 without the test compound to examine the extent of modulation. Control samples (untreated with candidate compounds) are assigned a relative B7S1 activity. Inhibition or activation is achieved when the measurement of T-cell activations deviates from about 10%, optionally 25%, 50%, or over 100% from the that of the control. The deviation, may be either an increase or decrease relative to the control, depending on the endpoint measured.

Modulators

The compounds tested as modulators of B7S1 can be any small chemical compound, or a biological entity, e.g., a macromolecule such as a protein, sugar, nucleic acid or lipid. Alternatively, modulators can be genetically altered versions of B7S1. Typically, test compounds will be small chemical molecules, antibodies, or nucleic acids. Essentially any chemical compound can be used as a potential modulator in the assays of the invention. Most often, organic compounds can be dissolved in aqueous or organic (especially DMSO-based) solutions. The assays are designed to screen large chemical libraries by automating the assay steps, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide, e.g, antibody, library containing a large number of potential therapeutic compounds (potential modulators). Such libraries are then screened in one or more assays, as described herein, to identify those library members (particular chemical species or subclasses) that display a desired characteristic activity. Any of the assays for detecting B7S1 activity are amenable to high throughput screening. High throughput assays binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Russell & Sambrook, all supra), peptide nucleic acid libraries (see, e.g., U.S. Pat. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

In some embodiments, the agents, e.g., small organic molecules, have a molecular weight of less than 1,500 daltons, and in some cases less than 1,000, 800, 600, 500, or 400 daltons. The relatively small size of the agents can be desirable because smaller molecules have a higher likelihood of having physiochemical properties compatible with good pharmacokinetic characteristics, including oral absorption than agents with higher molecular weight. For example, agents less likely to be successful as drugs based on permeability and solubility were described by Lipinski et al. as follows: having more than 5 H-bond donors (expressed as the sum of OHs and NHs); having a molecular weight over 500; having a LogP over 5 (or MLogP over 4.15); and/or having more than 10 H-bond acceptors (expressed as the sum of Ns and Os). See, e.g., Lipinski et al. Adv Drug Delivery Res 23:3-25 (1997). Compound classes that are substrates for biological transporters are typically exceptions to the rule.

In one embodiment the invention provides soluble assays using molecules such as a domain, e.g., an extracellular domain. In another embodiment, the invention provides solid phase based in vitro assays in a high throughput format, where the domain, full length B7S1 polyppetide, or cell expressing a B7S1 is attached to a solid phase substrate.

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (eg., the B7S1 of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and are appropriate tag binders; see, SIGMA Immunochemicals 1998 catalogue SIGMA, St. Louis Mo.).

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

In some embodiments, antibody or peptide libraries may be screened for the ability to bind to B7S1. Various “display libraries” are known to those of skill in the art and include libraries such as phage, phagemids, yeast and other eukaryotic cells, bacterial display libraries, plasmid display libraries as well as in vitro libraries that do not require cells, for example ribosome display libraries or mRNA display libraries, where a physical linkage occurs between the mRNA or cDNA nucleic acid, and the protein encoded by the mRNA or cDNA. Antibodies or peptides can further be assessed for the ability to modulate B7S1-medicated inhibition of T-cell activation.

Computer-Based Assays

Yet another assay for compounds that modulate B7S1 activity involves computer assisted drug design, in which a computer system is used to generate a three-dimensional structure of B7S1 based on the structural information encoded by the amino acid sequence. The input amino acid sequence interacts directly and actively with a pre-established algorithm in a computer program to yield secondary, tertiary, and quaternary structural models of the protein. The models of the protein structure are then examined to identify the regions that have the ability to bind, e.g., to blocking antibodies or to activated T-cells. These regions can be used to identify various compounds that modulate B7S1 binding or activity. For example, computer molecules may be used to design or identify potential agonist (or anatagonist) candidate compounds. These molecules can then be tested in a T-cell activation assay.

Computer systems are also used to screen for mutations, polymorphic variants, alleles and interspecies homologs of B7S1 genes. Such mutations can be associated with disease states or genetic traits. Once the variants are identified, diagnostic assays can be used to identify patients having such mutated genes or a propensity to have a particular disease or condition.

Expression Assays

Certain screening methods involve screening for a compound that modulates the expression of B7S1. Such methods generally involve conducting cell-based assays in which test compounds are contacted with one or more cells expressing B7S1 and then detecting an increase or decrease in expression (either transcript or translation product). Such assays are typically performed with cells that express endogenous B7S1.

Expression can be detected in a number of different ways. As described herein, the expression levels of the protein in a cell can be determined by probing the mRNA expressed in a cell with a probe that specifically hybridizes with a B7S1 transcript (or complementary nucleic acid derived therefrom). Alternatively, protein can be detected using immunological methods in which a cell lysate is probed with antibodies that specifically bind to the B7S1 protein.

Kits

B7S1 polypeptides e.g., recombinant B7S1 polypeptides, and antibodies are a useful tool for identifying cells such as activated T-cells or antigen-presenting cells and for diagnosing and treating immune system related disease.

The present invention also provides for kits for screening for modulators of B7S1 activity. Such kits can be prepared from readily available materials and reagents. For example, such kits can comprise any one or more of the following materials: a B7S1, typically a recombinant B7S1, reaction tubes, and instructions for testing B7S1 activity. Optionally, the kit contains biologically active B7S1. A wide variety of kits and components can be prepared according to the present invention, depending upon the intended user of the kit and the particular needs of the user.

Disease Treatment and Diagnosis

In certain embodiments, B7S1 sequences or modulators can be used in the diagnosis and treatment of certain diseases or conditions, i.e., immune-associated disorders. For example, B7S1 inhibitors can be used to enhance immune response, e.g., for treatment of cancer or infectious disease.

Further, B7S1 inhibits T-cell activation. It is preferentially expressed in antigen-presenting cells, e.g., macrophages, bone marrow, and B-cells Thus, mimics of B7S1 activity, i.e., compounds that enhance B7S1 activity or have the same activity, may be used to treat diseases or conditions associated with heightened immune system and inflammatory responses, e.g., autoimmune disease, vascular disease, and various malignancies of the immune system (see, e.g., Harrison's Principles of Internal Medicine, 12th Edition, Wilson, et al., eds., McGraw-Hill, Inc.). For example, syndromes that include an immune and/or inflammatory component include chronic inflammatory diseases including, but not limited to, connective tissue disorders, e.g., osteoarthritis, multiple sclerosis, Guillain-Barre syndrome, Crohn's disease, inflammatory bowel disease, ulcerative colitis, psoriasis, graft versus host disease, systemic lupus erythematosus, autoimmune thyroiditis, allergies, and insulin-dependent diabetes mellitus. Other inflammatory and autoimmune diseases include diseases due to oxidative or ischemic injury, e.g., damage to blood vessels; atherosclerosis, asthma, inflammation of the skin, eyes, or joints, e.g., ankylosing spondylitis, psoriasis, sclerosing cholangitis; and other autoimmune diseases (see, e.g., Harrison's Principles of Internal Medicine, supra).

Further, dysfunction in B7S1 may produce a disease, condition, or symptom associated with immune responses. Thus, mutation or dysregulation of the polypeptide could lead to disorders involving the immune response. Thus, in instances where there is a dysfunction, B7S1 sequences may therefore be used to detect, or diagnose a propensity for, these various immune -associated disorders.

Administration and Pharmaceutical Compositions

Modulators, e.g., antibodies, peptides, small organic molecules, of B7S1 activity can be administered to a mammalian subject for modulation of T-cell activation in vivo, e.g., for the treatment of any of the diseases or conditions described supra. As described in detail below, the modulators are administered in any suitable manner, optionally with pharmaceutically acceptable carriers.

The identified modulators can be administered to a patient at therapeutically effective doses to prevent, treat, or control diseases and disorders mediated, in whole or in part, by B7S1. The compositions are administered to a patient in an amount sufficient to elicit an effective protective or therapeutic response in the patient. An amount adequate to accomplish this is defined as “therapeutically effective dose.” The dose will be determined by the efficacy of the particular B7S1 modulators employed and the condition of the subject, as well as the body weight or surface area of the area to be treated. The size of the dose also will be determined by the existence, nature, and extent of any adverse effects that accompany the administration of a particular compound or vector in a particular subject.

Toxicity and therapeutic efficacy of such compounds can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

Pharmaceutical compositions for use in the present invention can be formulated by standard techniques using one or more physiologically acceptable carriers or excipients. The compounds and their physiologically acceptable salts and solvates can be formulated for administration by any suitable route, including via inhalation, topically, nasally, orally, parenterally (e.g., intravenously, intraperitoneally, intravesically or intrathecally) or rectally.

For oral administration, the pharmaceutical compositions can take the form of, for example, tablets or capsules prepared by conventional means with pharmaceutically acceptable excipients, including binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

Nucleic Acid Inhibitors of Gene Expression

In one aspect of the present invention, inhibitors of B7S1 can comprise nucleic acid molecules that inhibit expression of B7S1. Conventional viral and non-viral based gene transfer methods can be used to introduce nucleic acids encoding engineered polypeptides, e.g., dominant negative forms of the protein, in mammalian cells or target tissues, or alternatively, nucleic acids e.g., inhibitors of target protein expression, such as siRNAs, anti-sense RNAs, or ribozymes. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

In some embodiments, small interfering RNAs are administered. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNA inhibitors are less than 100 base pairs, typically 30 bps or shorter, and are made by approaches known in the art. Exemplary siRNAs according to the invention can have up to 29 bps, 25 bps, 22 bps, 21 bps, 20 bps, 15 bps, 10 bps, 5 bps or any integer thereabout or therebetween.

Non-Viral Delivery Methods

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Viral Delivery Methods

The use of RNA or DNA viral based systems for the delivery of inhibitors B7S1 are known in the art. Conventional viral based systems for the delivery of such nucleic acid inhibitors can include retroviral, lentivirus, adenoviral, adeno-associated and herpes simplex virus vectors for gene transfer.

In many gene therapy applications, it is desirable that the gene therapy vector be delivered with a high degree of specificity to a particular tissue type, e.g., B-cells, or other antigen presenting cells. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient.

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

Use of B7S1 Polypeptides or Mimics of B7S1 Activity

In some embodiments, it may be desirable to administer a B7S1 polypeptide in order to inhibit B7S1 activity, e.g., in automimmune disease. Thus, B7S1 proteins or domains thereof can be administered to reduce T-cell activation. Such polypeptide compositions can include, e.g.,encapsulated peptide compositions, e.g., poly(D,L-lactide-co-glycolide, “PLG”), microspheres (see, e.g., Eldridge, et al. (1991) Molec. Immunol. 28:287-294; Alonso, et al. (1994) Vaccine 12:299-306; Jones, et al. (1995) Vaccine 13:675-681), and comprise various pharmaceutical components.

The polypeptides can also be administered via nucleic acid compositions wherein DNA or RNA encoding a B7S1 polypeptide, or a fragment thereof, is administered to a patient. The techniques by which these are administered include those described above relating to inhibitors. Examples include “naked DNA” delivery, cationic lipid complexes, particle-mediated (“gene gun”) or pressure-mediated delivery, and the use of various viral or bacterial vectors. A wide variety of viral and bacterial vectors useful for therapeutic administration are are available, e.g., adeno and adeno-associated virus vectors, retroviral vectors, Vaccinia virus vectors, Salmonella typhi vectors, detoxified anthrax toxin vectors, and the like. See, e.g., Shata, et al. (2000) Mol. Med. Today 6:66-71; Shedlock, et al. (2000) J. Leukoc. Biol. 68:793-806; and Hipp, et al. (2000) In Vivo 14:571-85.

### EXAMPLES

### Example 1

### Identification of B7S1 as a New Member of the B7 Family

To identify novel members of the B7 family with potential function in immune regulation, a homology search in mouse and human EST databases using amino acid sequences of B7h and B7-H3 was performed. Human FLJ22418 molecule was found to share significant homology with these two B7-like molecules (data not shown). In addition, a mouse EST clone was found to contain nucleotide sequence encoding amino acids that are similar to the most N-terminus of FLJ22418. At the time, no function had been attributed to these sequences. A mouse B7S1 EST clone was obtained from Incyte and completely sequenced. The deduced peptide sequence from the mouse open reading frame shares striking homology with the human protein (FIG. 1B). They contain an N-terminal hydrophobic region which can serve as a leader peptide, two immunoglobulin (Ig)-like domains, and a hydrophobic C-terminus. Therefore, this novel protein shares common structural features with known members of the B7 family. The Ig-like domains of the deduced B7S1 protein were predicted by CD-Search program of NCBI. The alignment of mouse and human B7S1 protein sequences was performed using Jellyfish software, Biowire. The phylogeny tree was constructed with GeneWorks software. In a phylogenic analysis, B7S1 was most similar to B7h and B7-H3 (FIG. 1C).

### Example 2

### Construction of B7S1-Ig Fusion Protein

The nucleotide sequence encoding the extracellular portion of the mouse B7S1 molecule was amplified and cloned into the DES-Ig insect expression vector. This new plasmid was stably transfected into the S2 Drosophila cells which can be induced to secrete large amounts of B7S1-Ig fusion proteins. B7S1-Ig protein produced in this fashion was then purified by a Protein A column.

### Example 3

### Generation of Anti-B7S1 Monoclonal Antibodies

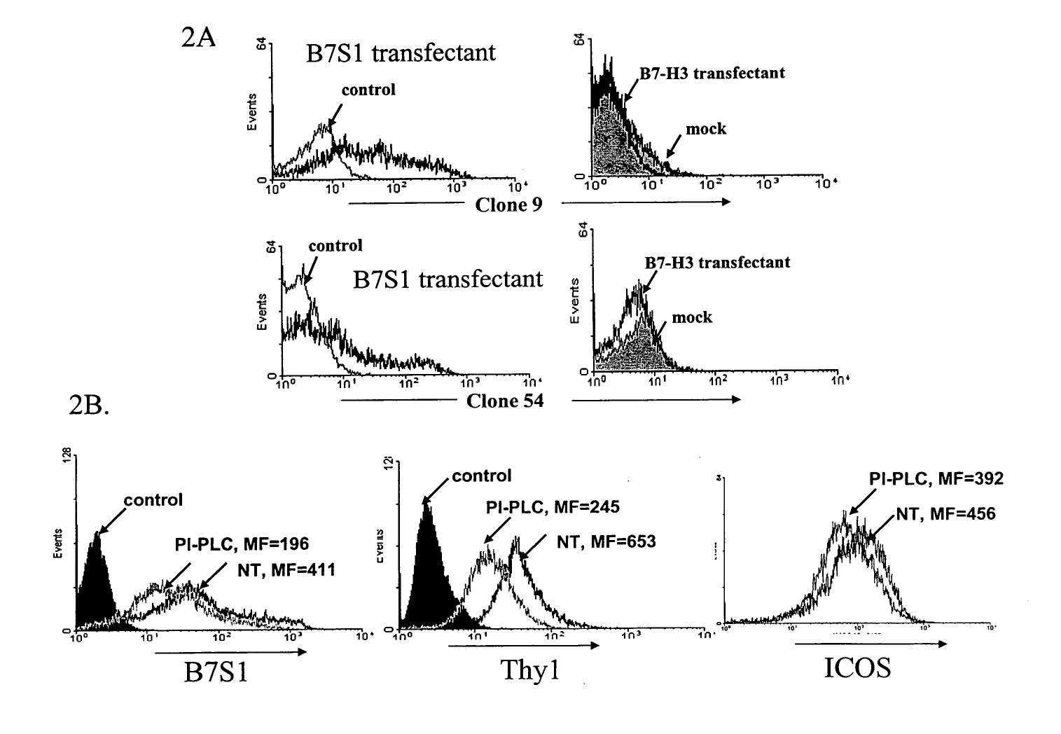

A female Lewis rat (3-4 month old) was immunized with 100 μg B7S1-Ig in complete Freud's adjuvant (CFA) at the foodpad, axial and lignguinal areas and boosted every 3^{rd} day in the same protein quantity once with antigen in IFA and 4 times with antigen in PBS. The draining lymph node cells were harvested 1 day after the last boost and fused with Ag8.653 cells by PEG1500. ELISA was performed to identify the clones which produced IgG antibodies reacting with B7S1-Ig fusion protein but not with control human IgG1. These clones were further subcloned. Three IgG monoclonal antibodies were generated which stained with different affinities 293 cells transfected with a mouse B7S1 expression vector (FIG. 2A) but not the mock transfectant (FIG. 2B). At least one of them, the clone 9 antibody, appears to be specific for B7S1 as it did not bind to 293 cells transfected with a B7-H3 expression plasmid (FIG. 2B).

### Example 4

### Flow Cytometry Analysis of B7S1 Expression

B7S1-Ig and anti-B7S1 were purified by protein A and G, respectively and biotinylated with Sulfo-NHS-LC-Biotin (Pierce). These reagents were used in conjunction with anti-CD4, CD8, CD11b, CD11c and B220 antibodies (Pharmingen) for analysis of various populations of immune cells by flow cytometry. The cells were pre-blocked using for non-specific binding with a human IgG1 before staining cells with B7S1-Ig.

### Example 5

### B7S1 expression is sensitive to PI-PLC treatment

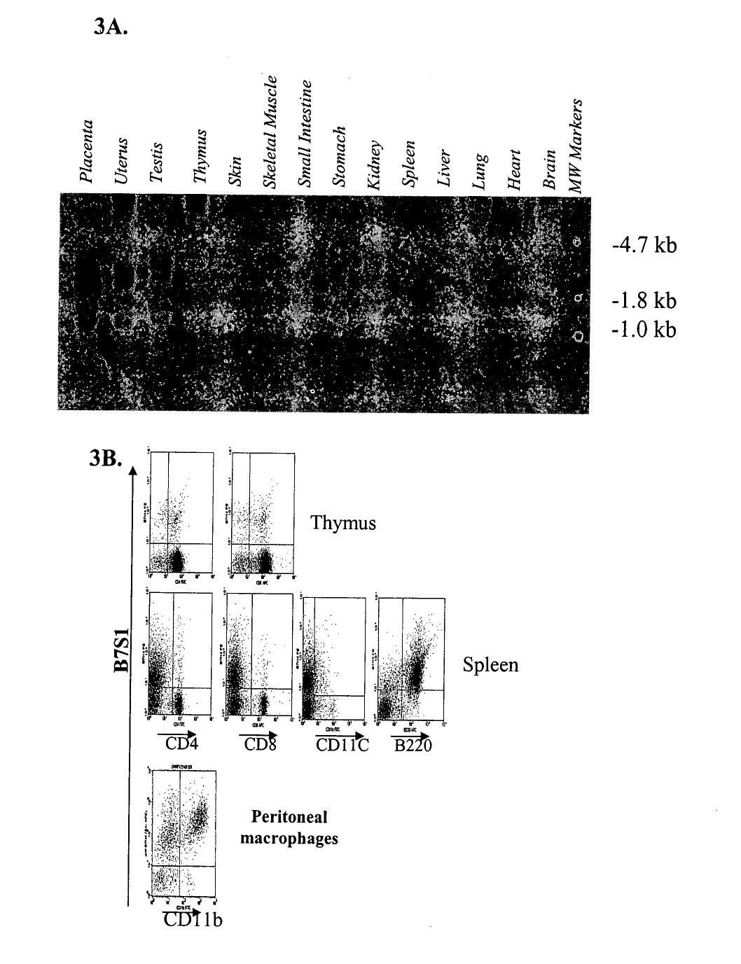

Although the C-terminus of the B7S1 protein is hydrophobic, it is not followed by any residues that can be predicted as intracellular amino acids. This structural characteristic is usually shared by cell surface GPI-anchored proteins. We therefore tested whether cell surface expression of B7S1 is sensitive to phosphatidylinositol-specific phospholipase C (PI-PLC) treatment. 293 cells transfected with a B7S1 expression vector and EL4 cells were treated with PI-PLC (Sigma) in Hank's solution for 30 minutes at 37C, followed by flow cytometry analysis. Indeed, PI-PLC reaction resulted in >50% reduction of B7S1 expression on transfected 293 cells (FIG. 3). As a positive control, expression of GPI-anchored Thy1 molecule on EL-4 can be reduced to a similar extent by PI-PLC while transmembrane protein ICOS is largely insensitive (FIG. 3). B7S1 thus represents the first GPI-linked protein in the B7 family.

### Example 6

### Expression of B7S1 Molecule by Professional APC

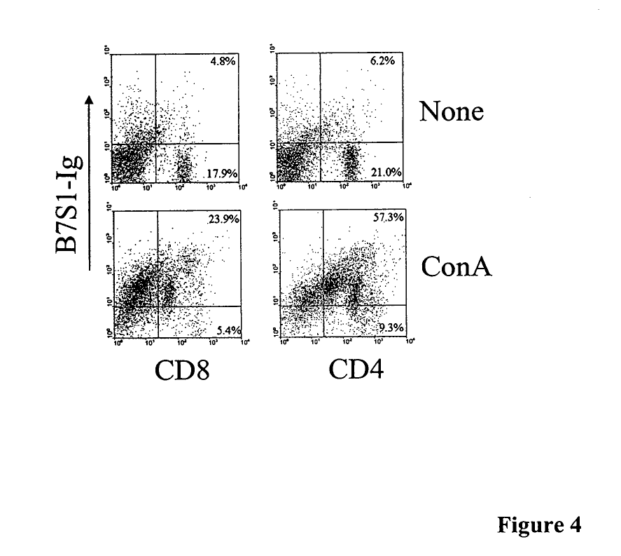

All known members of the B7 family are expressed by professional APC. In addition, the new members of this family, i.e. B7h, PDL1, PDL2 and B7-H3 are broadly distributed in non-lymphoid tissues and cells. To assess the possible immune regulation by B7S1, we first examined the expression of B7S1 mRNA by a Northern blot analysis. A cDNA probe consisting of the coding sequence for the extracellular region of B7S1 was utilized. B7S1 expression is detected in lymphoid tissues thymus and spleen, and in a number of non-lymphoid organs except liver (FIG. 4A). Ubiquitous expression of B7S1 in non-lymphoid tissues is indicative of its role in modulating immune responses in these tissues.

B7S1 expression in immune cells was further examined using the anti-B7S1 monoclonal antibody. In thymus, B7S1 is expressed by a minor population of cells which also express CD4 and CD8 (FIG. 4B). In spleen, B7S1 is not expressed by CD4+, CD8+ or CD11C+ cells, but is constitutively on all B cells (FIG. 4B). Therefore, in second lymphoid organs, B7S1 exhibits B cell-specific expression. However, B7S1 expression is detected on peritoneal CD11b+ macrophages elicited by thioglycollate and on bone marrow-derived CD11C+ dendritic cells (data not shown). This analysis indicated that B7S1 is expressed by a variety of professional APC and possesses regulatory roles on T cells.

Members of the B7 family are differentially regulated in professional APC by various stimuli. For instance, CD80 and CD86 expression can be induced by innate activation; B7h is downregulated on B cells by IgM engagement and upregulated on fibroblasts by TNFα. B7S1 regulation in APC was therefore examined. LPS treatment of peritoneal macrophages or bone marrow-derived dendritic cells did not significantly alter the B7S1 expression (data not shown). On the other hand, multiple stimuli of purified splenic B cells, including LPS, IL-4, anti-IgM and anti-CD40, all resulted in 50-70% reduction of B7S1 expression (FIG. 4C). This result indicated that B7S1 is downregulated after B cell activation.

### Example 7

### T cell Activation and Differentiation

CD4+ T cells from C57BL/6, or OT-II mice were isolated. The cells were treated with plate-bound anti-CD3 in the absence or presence of human IgG1 (Sigma), B7.1-Ig (R&D) or B7S1-Ig. IL-2 production was measured 24 hours after T cell activation, and cell proliferation was measured 96 hours after the treatment with ³H-thymidine in the last 8 hours. CD4 T cell differentiation and restimulation of effector T cells were performed.

### Example 8

### Nuclear Extract Preparation and Immunoblotting Analysis

To determine the molecular target of B7S1, we purified nuclear extracts of T cells activated for 24 hours were and expression of AP-1, NFAT and NFKB transcription factors was analyzed by western blot analysis using antibodies from Santa Cruz.

### SUMMARY

B7S1-Ig acts as an agonistic agent mimicking B7S1 activity, to inhibit T cell function in immune diseases.

Anti-B7S1 acts as an antagonistic blocker of B7S1 function to enhance anti-microbial and anti-tumor immune responses.

Inhibition of T Cell Activation by B7S1-Ig

Expression of B7S1 on professional APC suggests a role of B7S1 in regulation of T cell immune responses. We employed our B7S1-Ig fusion protein to assess if B7S1 has a putative receptor on T cells. CD4+ and CD8+ T cells from C57BL/6 lymph nodes are not strongly bound by the biotinylated B7S1-Ig; after ConA activation for two days, all of them do (FIG. 4). B7S1-Ig binds to CD28−/− cells to the same degree (data not shown). B7.1-Ig does not block binding of B7S1-Ig to activated T cells (data not shown), suggesting that it does not recognize CD28 or CTLA4. In addition, B7S1-Ig binds well to ICOS −/− cells activated by ConA (data not shown). It does not stain 293 cells transfected with PD-1, which is recognized by an anti-PD-1 antibody (data not shown). B7-H3-Ig and B7S1-Ig does not reciprocally block the binding of the other fusion protein to their corresponding receptor on activated T cells (data not shown). All these data indicate a putative receptor for B7S1 on activated T cells, which is distinct from CD28, CTLA4, ICOS, PD-1 and the receptor for B7-H3.

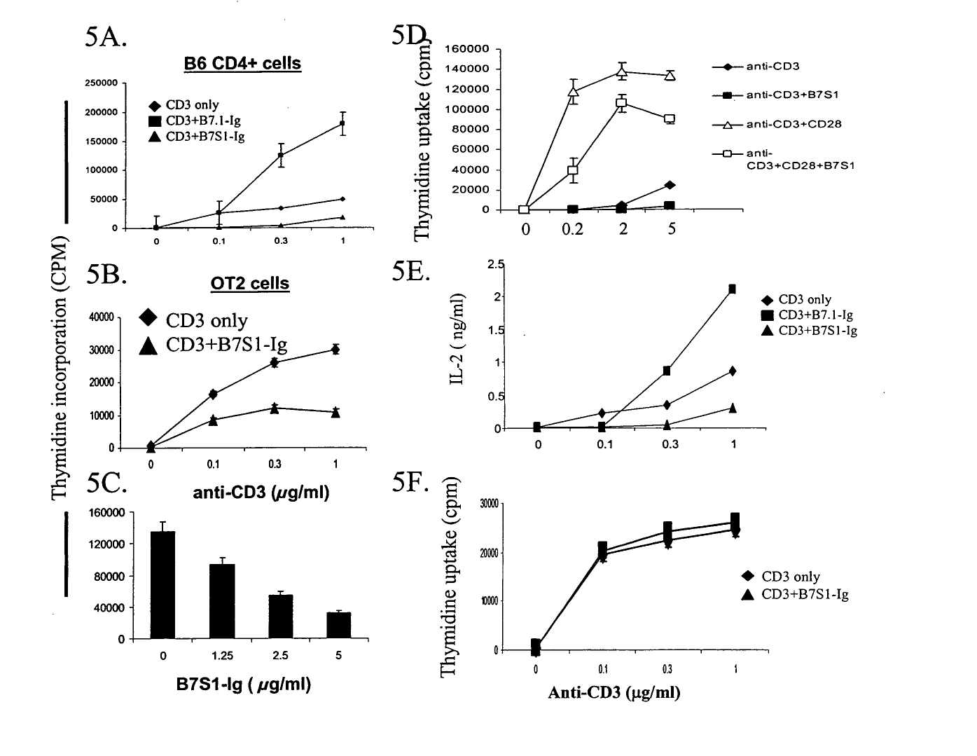

To assess the function of B7S1 on T cell activation and function, we stimulated purified CD4+ cells from C57BL/6 (FIG. 5A) or OT-II TcR transgenic (FIG. 5B) mice with different doses of anti-CD3 in the absence or presence of B7.1-Ig or B7S1-Ig and measured cell proliferation. Anti-CD3 plus a human 1 control results in the similar proliferation of stimulated T cells as the ones treated with anti-CD3 only (data not shown). B7.1-Ig, as expected, strongly enhances T cell stimulation (FIG. 5A). B7S1-Ig on the other hand, inhibits T cell proliferation (FIG. 5A-B). To rule out the possibility that this inhibitory effect is artificially derived from our insect culture system, we employed an irrelevant protein containing the human IgG1 tag expressed and prepared in the same fashion as B7S1-Ig and found it has no effect on T cell proliferation (data not shown). B7S1-Ig inhibits proliferation of OT-II transgenic T cells in a dose-dependent manner (FIG. 5C). B7S1-Ig treatment also moderately reduces CD25 and CD44 upregulation on activated OT-II T cells (data not shown). In the presence of CD28 costimulation, B7S1-Ig inhibits T cell proliferation, most potently when a low dose of anti-CD3 is used (FIG. 5D). Interestingly, strong TcR and CD28 costimulation partially overcomes this inhibition (FIG. 5D). These results indicate B7S1 is a negative regulator of T cell activation and B7S1-Ig treatment renders the cells less responsive to TcR and CD28 signaling.

The hallmark of T cell activation is the production of IL-2, which drives T cell clonal expansion. We thus examined whether IL-2 production is affected by B7S1-Ig costimulation. While B7.1-Ig strongly potentiates IL-2 production, B7S1-Ig inhibits (FIG. 5E). B7S1 -Ig also inhibits IL-2 production by cells treated with anti-CD3 and anti-CD28 (data not shown). To assess whether inhibition of T cell proliferation by B7S1-Ig is the result of IL-2 reduction, exogenous IL-2 was added to the OT-II T cells treated with anti-CD3 with or without B7S1-Ig. Addition of IL-2 fully restored the proliferation of T cells costimulated with B7S1-Ig (FIG. 5F). Therefore, B7S1 inhibits T cell activation via reducing the IL-2 production. Effector Th cells differentiated in this fashion exhibited no cytokine defect (data not shown). This shows that B7S1 inhibits T cell activation and IL-2 production.

IL-2 gene induction in activated T cells is the result of multiple signaling pathways from cell surface receptors leading to activation of NFAT, NF-KB and AP1 transcription factors. We assessed whether B7S1-Ig costimulation results in an inhibition of IL-2 transcription machinery. OT-II T cells were treated with anti-CD3 in the presence or absence of B7S1-Ig costimulation and nuclear extracts prepared 16 hours after stimulation. Nuclear localization of NFATc1 and c-rel transcription factors, both of which bind to the IL-2 promoter and are important for its induction, is the result of T cell activation. B7S1-Ig costimulation does not reduce the amount of these two factors in the nucleus as examined by western blotting (data not shown). However, the expression of JunB, a component of the AP-1 family induced after T cell activation, is reduced by 49% after B7S1 costimulation. On the other hand, there is little change in c-Jun expression (11% reduction) with B7S1-Ig treatment. JunB has been previously shown to bind to the IL-2 promoter and JunB overexpression results in greater IL-2 production. Since JunB is induced after T cell activation, B7S1 costimulation results in inefficient JunB induction. Overall, this analysis shows B7S1-Ig treatment leads to a selective signaling and transcription defect.

Enhanced T cell Activation by an Anti-B 7S1 Blocking Antibody

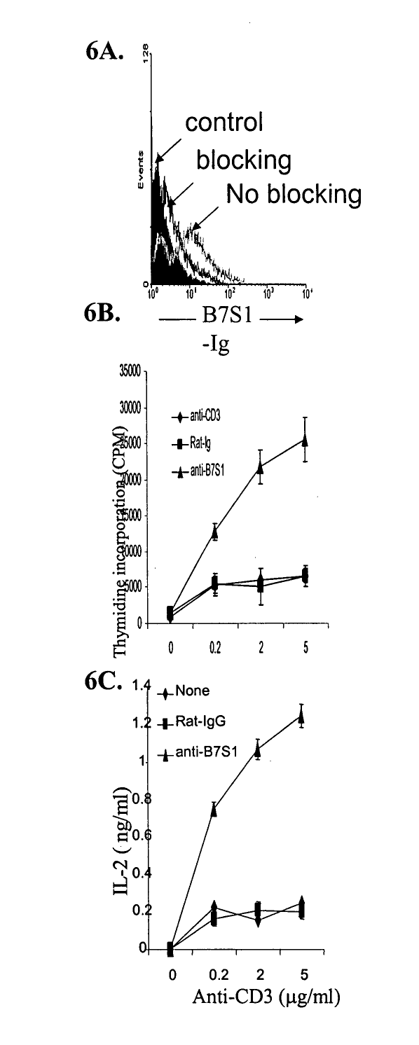

To assess the physiological importance of B7S1 binding to its corresponding receptor in immune responses, we employed an anti-B7S1 blocking antibody for our in vitro and in vivo analysis. We found that clone 54 antibody inhibits binding of B7S1-Ig to activated T cells (FIG. 6A), while clone 9 does not significantly (data not shown).

We first assessed the function of this blocking antibody in vitro by activating splenocytes from C57BL/6 mice with different doses of anti-CD3. In this experiment, positive and negative costimulation is provided by different splenic APCs, mostly B cells. While a control rat IgG does not alter the T cell proliferation, treatment with anti-B7S1 blocking antibody greatly enhances it (FIG. 6B). We also measured IL-2 production within first 24 hours of treatment, and find that B7S1 blocking antibody also greatly increases the levels of IL-2 production by T cells (FIG. 6C). This work substantiates the above data using B7S1-Ig and indicates that B7S1 is a physiological negative regulator of T cell activation and IL-2 expression.

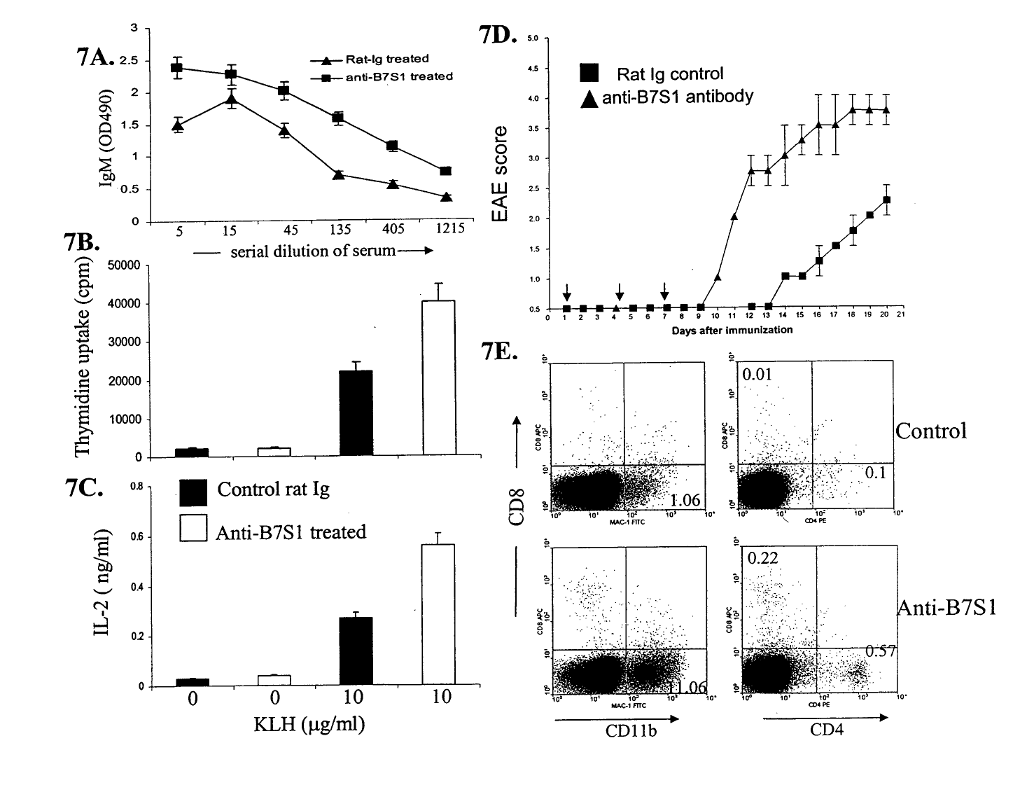

To examine the important role of negative regulation by B7S1 in immune function in vivo, we immunized C57BL/6 mice at their base of tail with KLH protein emulsified in CFA. A control rat IgG or the B7S1 blocking antibody was injected into experimental mice every other day for a total of 3 times. 8 days after the immunization, the mice were sacrificed and anti-KLH antibody in the serum was measured. We found that treatment with anti-B7S1 blocking antibody leads to greater anti-KLH IgM (FIG. 7A) and IgG (data not shown) production, indicative of a stronger immune response in vivo. We also collected spleen cells from immunized mice and restimulated them in vitro with or without 10 μg/ml KLH. Cells from anti-B7S1 treated mice consistently exhibit greater proliferation and IL-2 production (FIG. 7B-C), also demonstrating that stronger T cell priming occurs in vivo in the presence of anti-B7S1 blocking antibody.

To assess the importance of B7S1 in T cell activation and tolerance, we immunized C57BL/6 with MOG35-55 peptide to induce EAE disease. Control or anti-B7S1 blocking antibody was injected into mice during T cell priming phase, i.e. between first and second immunization. Mice treated with anti-B7S1 blocking antibody consistently develop greatly accelerated and much more robust EAE than those treated with a control rat antibody (FIG. 7D). When we examined infiltrating mononuclear cells in the brain of experimental mice, we found that anti-B7S1 antibody treatment in mice results in greater CD4 and CD8 cell infiltration, and also increases CD11b+ macrophages (FIG. 7E). This work strongly demonstrates an important function of B7S1 in negative regulation of T cell activation.

### CONCLUSION

Antagonistic blockers, such as but not limited to anti-B7S1, increase anti-microbial and anti-tumor immune responses for treatment of diseases where the immune system needs to be enhanced such as, but not limited to infectious diseases and tumor immunotherapy. We have examined the physiological significance of B7S1 costimulation by use of a blocking antibody. Treatment of anti-CD3-activated splenocytes with this antibody greatly enhanced IL-2 production and T cell proliferation (FIG. 6B-C). This work, in agreement with our results using B7S1-Ig (FIG. 5A-F), indicates that B7S1 expressed on APC does function physiologically to limit the amount of IL-2 expression by activated T cells and hence the extent of their clonal expansion. More importantly, blocking of B7S1 function led to greater T cell priming and function in vivo. In an immunization experiment, we found that treatment of immunized mice with anti-B7S1 blocking antibody led to greater antigen-specific Ig production (FIG. 7A). Furthermore, enhanced IL-2 production and T cell proliferation by restimulated spleen cells harvested from anti-B7S1 treated mice also support that T cell priming is indeed enhanced in vivo (FIG. 7B-C). Thus blocking B7S1 possesses potential therapeutic value in enhancing wanted immune responses.

B7S1, like other B7 family members, possesses one pair of Ig-like domains in its extracellular region (FIG. 1B). However, it lacks an obvious transmemrbrane region. We show that its cell surface expression is sensitive to PI-PLC treatment (FIG. 3) and conclude that B7S1 is anchored to the cell membrane via a GPI linkage. Thus B7S1 is the first GPI-linked protein in the B7 family, and the rest are type I transmembrane glycoproteins. With a GPI linkage, B7S1 may be in close proximity to the MHC and this spatial organization may allow efficient negative costimulation to occur. This knowledge can be used to generate more efficient therapeutics by designing molecules in which the linkage is optimized for the desired effect.

With the monoclonal antibodies we generated against mouse B7S1 molecule, we found B7S1 is expressed by most professional APC, including bone marrow-derived dendritic cells, peritoneal macrophages and B cells. Its expression on B cells is downregulated by multiple stimuli (FIG. 4C). This observation demonstrates costimulatory regulation of T cells by B7S1 is influenced by the activation status of B cells. It is noteworthy that other B7 family members are regulated differentially in B cells. CD80 and CD86 are well known to be upregulated after B cell activation while B7h is downregulated only after IgM crosslinking. All these suggest a combinatorial model for costimulation of T cells: each B7 ligand is regulated differentially, which reflects the natural history of APC, and the combination of these ligands regulates the threshold of T cell activation. On the other hand, the new B7 family members- B7h, PDL1/2, B7-H3 and B7S1 are also widely distributed. Since their receptors are expressed on activated T cells they may possess important function in modulating effector T cell function once activated T cells migrate into the non-lymphoid tissues. It is also possible that at this effector stage, the combinatorial signals presented by B7 ligands which are tissue-specific and regulated by inflammatory cytokines may influence the nature and extent of T cell function. This invention allows further understanding of the combinatorial signals, such that they can be manipulated for maximum therapeutic benefit in the design of treatment regimens.

A B7S1-Ig fusion protein we prepared bound to activated but not naïve CD4 and CD8 cells. Therefore, B7S1 joins B7h, PDL1/2 and B7-H3 to recognize receptors induced after T cell activation. B7S1-Ig can bind to CD28 −/− and ICOS −/− cells; it does not react with a PD-1 transfectant. Also with the fusion protein, we tested the function of B7S1 on T cell activation and differentiation. B7S1-Ig very potently inhibits CD4+ T cell proliferation (FIG. 6A). We further show that B7S1 reduction of T cell proliferation is through an IL-2-dependent mechanism. B7S1-Ig greatly reduces the IL-2 production by activated T cells and addition of exogenous IL-2 restores the proliferation of T cells by B7S1-Ig-treated cells (FIG. 7A-B). These data demonstrate that B7S1 is a negative regulator of T cell activation and IL-2 production. We show that JunB induction is selectively reduced when T cells are costimulated by B7S1, indicating a mechanism of inhibition by B7S1 (FIG. 7C). JunB is induced after T cell activation and functions to regulate IL-2 gene transcription. It is interesting that B7S1 at this stage does not globally inhibit all signaling pathways but instead selectively blocks a specific mechanism. The JunB signaling pathway represents another target for therapeutic intervention of immune related disease.

We further assessed the importance of B7S1 negative costimulation using an autoimmunity mouse model- MOG-induced EAE in C57BL/6 mice. Anti-B7S1 blocking antibody treatment leads to an extreme EAE disease. It is noteworthy that the antibody was injected into experimental mice at T cell priming phase, i.e. after the first immunization but before the second. The disease normally becomes observable only after the second immunization. Therefore, anti-B7S1 effect is due to the enhanced expansion of autoreactive T cells. We find greater CD4+ and CD8+ T cell infiltration into the brain tissue in mice treated with anti-B7S1 antibody (FIG. 7E). Macrophages are also greatly increased in number in these mice as well. These results demonstrate an important role of B7S1 in negative regulation of T cell-dependent autoimmune reaction. Thus, the primary cause of this greatly enhanced autoimmunity is primarily due to excessive T cell activation and clonal expansion in vivo. B7S1 is additionally important to inhibit certain effector or to generate regulatory T cells in vivo that function to contain autoimmunity. The EAE experiment indicates that B7S1 contributes to the maintenance of peripheral tolerance and to the containment of autoimmune diseases, and manipulation of B7S1 may be used therapeutically to modulate these responses.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

Human and mouse B7S1 nucleic acid and polypeptide sequences:

SEQ ID NO:1 human B7S1 nucleic acid sequence open
reading frame
atggcttccc tggggcagat cctcttctgg agcataatta
gcatcatcat tattctggct ggagcaattg cactcatcat
tggctttggt atttcaggga gacactccat cacagtcact
actgtcgcct cagctgggaa cattggggag gatggaatcc
agagctgcac ttttgaacct gacatcaaac tttctgatat
cgtgatacaa tggctgaagg aaggtgtttt aggcttggtc
catgagttca aagaaggcaa agatgagctg tcggagcagg
atgaaatgtt cagaggccgg acagcagtgt ttgctgatca
agtgatagtt ggcaatgcct ctttgcggct gaaaaacgtg
caactcacag atgctggcac ctacaaatgt tatatcatca
cttctaaagg caaggggaat gctaaccttg agtataaaac
tggagccttc agcatgccgg aagtgaatgt ggactataat
gccagctcag agaccttgcg gtgtgaggct ccccgatggt
tcccccagcc cacagtggtc tgggcatccc aagttgacca
gggagccaac ttctcggaag tctccaatac cagctttgag
ctgaactctg agaatgtgac catgaaggtt gtgtctgtgc
tctacaatgt tacgatcaac aacacatact cctgtatgat
tgaaaatgac attgccaaag caacagggga tatcaaagtg
acagaatcgg agatcaaaag gcggagtcac ctacagctgc
taaactcaaa ggcttctctg tgtgtctctt ctttctttgc
catcagctgg gcacttctgc ctctcagccc ttacctgatg
ctaaaataa

SEQ ID NO:2 human B7S1 polypeptide sequence
MASLGQILFWSIISIIIILAGAIALIIGFGISGRHSITVTTVASAGNIGE
DGIQSCTFEPDIKLSDIVIQWLKEGVLGLVHEFKEGKDELSEQDEMFRGR
TAVFADQVIVGNASLRLKNVQLTDAGTYKCYIITSKGKGNANLEYKTGAF
SMPEVNVDYNASSETLRCEAPRWFPQPTVVWASQVDQGANFSEVSNTSFE
LNSENVTMKVVSVLYNVTINNTYSCMIENDIAKATGDIKVTESEIKRRSH
LQLLNSKASLCVSSFFAISWALLPLSPYLMLK

SEQ ID NO:3 mouse B7S1 nucleic acid sequence open
reading frame
atggcttcct tggggcagat catcttttgg agtattatta
acatcatcat catcctggct ggggccatcg cactcatcat
tggctttggc atttcaggca agcacttcat cacggtcacg
accttcacct cagctggaaa cattggagag gacgggaccc
tgagctgcac ttttgaacct gacatcaaac tcaacggcat
cgtcatccag tggctgaaag aaggcatcaa aggtttggtc
cacgagttca aagaaggcaa agacgacctc tcacagcagc
atgagatgtt cagaggccgc acagcagtgt ttgctgatca
ggtggtagtt ggcaatgctt ccctgagact gaaaaacgtg
cagctcacgg atgctggcac ctacacatgt tacatccgct
cctcaaaagg caaggggaat gcaaaccttg agtataagac
cggagccttc agtatgccag agataaatgt ggactataat
gccagttcag agagtttacg ctgcgaggct cctcggtggt
tcccccagcc cacagtggcc tgggcatctc aagttgacca
aggagccaac ttctcagaag tctccaacac cagctttgag
ttgaactctg agaatgtgac catgaaggtc gtatctgtgc
tctacaatgt cacaatcaac aacacatact cctgtatgat
tgaaaacgac attgccaaag ccaccgggga catcaaagtg
acagattcag aggtcaaaag gcggagtcag ctgcagttgc
tgaactctgg gccttccccg tgtgtttctt cttctgcctt
tgtggctggc tgggcactcc tatctctctc ctgttgcctg
atgctaagat ga

SEQ ID NO:4 mouse B7S1 polypeptide sequence
MASLGQIIFWSIINIIIILAGAIALIIGFGISGKHFITVTTFTSAGNIGE
DGTLSCTFEPDIKLNGIVIQWLKEGIKGLVHEFKEGKDDLSQQHEMFRGR
TAVFADQVVVGNASLRLKNVQLTDAGTYTCYIRSSKGKGNANLEYKTGAF
SMPEINVDYNASSESLRCEAPRWFPQPTVAWASQVDQGANFSEVSNTSFE
LNSENVTMKVVSVLYNVTINNTYSCMIENDIAKATGDIKVTDSEVKRRSQ
LQLLNSGPSPCVSSSAFVAGWALLSLSCCLMLR

What is claimed is:

1. A method of enhancing T-cell activation, the method comprising contacting a T-cell with an agent that inhibits binding of B7S1 to the T-cell.

2. The method of claim 1, wherein the agent is an antibody.

3. The method of claim 2, wherein the antibody specifically binds B7S1 protein.

4. The method of claim 2, wherein the antibody is a monoclonal antibody.

5. The method of claim 2, wherein the antibody is a chimeric antibody.

6. The method of claim 2, wherein the antibody is a humanized antibody.

7. The method of claim 2, wherein the antibody is a human antibody.

8. The method of claim 2, wherein the antibody is a single chain Fv fragment (scFv).

9. The method of claim 1, wherein the agent is administered to a patient having an infectious disease or cancer.

10. A method of inhibiting T-cell activation, the method comprising administering a polypeptide comprising an amino acid sequence: (a) having at least 90% identity to amino acid residues 43-254 SEQ ID NO:2; or (b) comprising at least 100 contiguous amino acid residues of amino acids 43-254 of SEQ ID NO:2.

11. The method of claim 10, wherein the method comprises administering a polypeptide comprising amino acid residues 43-254 SEQ ID NO:2.

12. The method of claim 11, wherein the polypeptide is B7S1-Ig.

13. The method of claim 10, wherein the polypeptide is administered to a patient having an autoimmune disease.
